(19) United States
(12) Patent Application Publication
(10) Pub. No.: US 2023/0160907 A1
(43) Pub. Date: May 25, 2023

(54) CELL-BASED METHODS FOR PREDICTING POLYPEPTIDE IMMUNOGENICITY

(71) Applicant: GENENTECH, INC., South San Francisco, CA (US)

(72) Inventors: Lynn Anne Kamen, South San Francisco, CA (US); Rachel Ann Melendez, South San Francisco, CA (US); Shan Chung, South San Francisco, CA (US)

(73) Assignee: GENENTECH, INC., South San Francisco, CA (US)

(21) Appl. No.: 18/153,259

(22) Filed: Jan. 11, 2023

Related U.S. Application Data
(63) Continuation of application No. PCT/US21/41422, filed on Jul. 13, 2021.
(60) Provisional application No. 63/071,535, filed on Aug. 28, 2020.
(60) Provisional application No. 63/051,157, filed on Jul. 13, 2020.

(51) Int. Cl. G01N 33/68; G01N 33/50

(52) U.S. Cl. CPC G01N 33/6854; G01N 33/5047

(57) ABSTRACT

The presently disclosed subject matter provides methods for determining the propensity of a polypeptide or a fragment thereof, e.g., an antibody or a fragment thereof, to elicit production of anti-drug antibodies (ADAs) and kits for performing such methods.

### CROSS-REFERENCE TO RELATED APPLICATIONS

This application is a continuation application of International Patent Application No. PCT/US2021/041422, filed Jul. 13, 2021, which claims priority to U.S. Provisional Application No. 63/051,157, filed Jul. 13, 2020, and U.S. Provisional Application No. 63/071,535, filed Aug. 28, 2020, the contents of each of which are incorporated by reference in their entireties, and to each of which priority is claimed.

### FIELD OF THE INVENTION

The present disclosure relates to methods for determining the propensity of a polypeptide to elicit production of anti-drug antibodies (ADAs) and kits for performing such methods.

### BACKGROUND

Polypeptide-based therapeutics (e.g., antibodies) have greatly improved the treatment of an increasing number of serious and difficult to treat diseases. Unfortunately, such therapeutics may elicit the production of anti-drug antibodies (ADAs) when administered to a patient. ADAs can have neutralizing effects on the therapeutic. These neutralizing effects can include limiting the activity of the therapeutic, increasing clearance of the therapeutic, and a potential reduction in overall clinical response attributable to administration of the therapeutic. In certain instances, the production of ADAs has also coincided with the occurrence of severe adverse events in patients, including hypersensitivity reactions and anaphylaxis.

Understanding the immunogenicity of a polypeptide-based therapeutic in the preclinical phase of drug development can improve the likelihood of success of the therapeutic in subsequent clinical phases. While immunogenic epitopes are generally predicted using in silico tools, several cell-based techniques have been developed to determine the immunogenic potential of preclinical therapeutic candidates. One such technique is called major histocompatibility complex (MHC) class II-associated peptide proteomics (MAPPs). MAPPs involves incubating a population of antigen-presenting cells (APCs), e.g., dendritic cells, with a polypeptide-based therapeutic of interest. The APCs will internalize and process the therapeutic into short peptides. The peptides are loaded onto MHC class II molecules and presented on the surface of the APC. Immunoprecipitating and analyzing these MHC-peptide complexes via liquid chromatography mass spectrometry (LC/MS) allows for identification of potentially immunogenic epitopes in the polypeptide-based therapeutic. Another technique for determining the immunogenic potential of a preclinical therapeutic candidate is the T cell proliferation assay. The T cell proliferation assay involves the detection of T cell proliferation after co-culture with APCs, e.g., dendritic cells, that have been incubated with the polypeptide-based therapeutic of interest. These techniques, however, are labor intensive, time consuming and require numerous pieces of high-cost equipment. Accordingly, there is a need in the art for a more time-efficient and cost-effective method for determining the propensity of a polypeptide-based therapeutic to elicit production of ADAs.

### SUMMARY

The present disclosure provides methods for determining the propensity of a polypeptide, or a fragment thereof, to elicit the production of anti-drug antibodies (ADAs) relative to a known reference. In certain embodiments, the methods disclosed herein can include (a) contacting an antigen presenting cell (APC) with the polypeptide, or fragment thereof; (b) measuring the amount of polypeptide, or fragment thereof, present on an outer surface of the APC; (c) measuring the total amount of polypeptide, or fragment thereof, associated with the APC; (d) calculating an internalization index value by subtracting the amount of polypeptide, or fragment thereof, bound to the outer surface of the APC measured in (b) from the total amount of polypeptide, or fragment thereof, associated with the APC measured in (c); and comparing the internalization index in (d) to a reference internalization index indicative of a known propensity to elicit the production of ADAs. In certain embodiments, the total amount of polypeptide, or fragment thereof, associated with the APC includes the amount of polypeptide, or fragment thereof, present on the outer surface of the APC and the amount of the polypeptide, or fragment thereof, present within the APC. In certain embodiments, when the internalization index value in (d) is greater than the reference internalization index, the polypeptide, or fragment thereof, has a greater propensity to elicit ADAs than the reference. In certain embodiments, when the internalization index value in (d) is less than the reference internalization index, the polypeptide, or fragment thereof, has a lesser propensity to elicit ADAs than the reference.

In certain embodiments, the polypeptide, or fragment thereof, is a peptide. In certain embodiments, the polypeptide, or fragment thereof, is a recombinant protein. In certain embodiments, the polypeptide, or fragment thereof, is an antibody or fragment thereof, e.g., a human, humanized or chimeric antibody. In certain embodiments, the polypeptide, or fragment thereof, e.g., an antibody or fragment thereof, is a single domain antibody. In certain embodiments, the polypeptide, or fragment thereof, e.g., an antibody or fragment thereof, is an antibody-drug conjugate (ADC).

In certain embodiments, the APC is selected from the group consisting of a dendritic cell, a macrophage, a monocyte and a B cell. In certain embodiments, the APC is a dendritic cell. For example, but not way of limitation, the dendritic cell is an immature dendritic cell. In certain embodiments, the immature dendritic cell is generated by differentiating monocytes isolated from a donor, e.g., a human donor. In certain embodiments, the isolated monocytes are differentiated in the presence of one or more of interleukin-4 (IL-4) and granulocyte-macrophage colony-stimulating factor (GM-CSF) to generate the immature dendritic cells.

In certain embodiments, the APC is contacted with the polypeptide, or fragment thereof, in the presence of an agent. For example, but not by way of limitation, the agent is selected from the group consisting of an inflammatory cytokine, prostaglandin E2 (PGE2), lipopolysaccharide (LPS) and a combination thereof. Non-limiting examples of inflammatory cytokines include TNFα, IL-6, IL-1β and combinations thereof. In certain embodiments, the agent is LPS.

In certain embodiments, measuring the total amount of polypeptide, or fragment thereof, associated with the APC can include (i) permeabilizing the APC, (ii) contacting the APC with a detection agent that binds to the polypeptide or fragment thereof and (iii) determining the amount of detection agent that is bound to the polypeptide or fragment thereof, present on the outer surface of the APC and within the APC to measure the total amount of polypeptide or fragment thereof, associated with the APC. In certain embodiments, measuring the amount of polypeptide or fragment thereof, present on the outer surface of the APC can include (i) contacting the APC with a detection agent that binds to the polypeptide or fragment thereof and (ii) determining the amount of detection agent that is bound to the polypeptide or fragment thereof, present on the outer surface of the APC to measure the amount of polypeptide or fragment thereof, present on the outer surface of the APC, wherein the APC is not permeabilized prior to contacting the APC with the detection agent. In certain embodiments, the detection agent is an antibody, e.g., an antibody conjugated to a fluorophore. In certain embodiments, the antibody is an anti-IgG antibody. In certain embodiments, determining the amount of detection agent that is bound to the polypeptide, or fragment thereof, is performed by flow cytometry.

The present disclosure further provides kits for performing any one of the methods disclosed herein. In certain embodiments, the kit includes one or more of the following: an APC; an agent; a detection agent; and a permeabilization agent. In certain embodiments, the agent is selected from the group consisting of an inflammatory cytokine, prostaglandin E2 (PGE2), lipopolysaccharide (LPS) and a combination thereof. In certain embodiments, the inflammatory cytokine is selected from the group consisting of TNFα, IL-6, IL-1β and a combination thereof. In certain embodiments, the agent is LPS. In certain embodiments, the detection agent is an antibody, e.g., an antibody conjugated to a fluorophore. In certain embodiments, the detection agent is an anti-IgG antibody. In certain embodiments, the permeabilization agent is saponin.

### BRIEF DESCRIPTION OF THE FIGURES

FIG. 1 displays a schematic of a non-limiting embodiment of a method for determining the propensity of a polypeptide to elicit production of ADAs.

FIG. 2 displays a schematic for detecting the amount of antibody bound to the surface of an antigen-presenting cell (APC) and the amount of total antibody associated with the APC.

FIG. 3 displays a flow cytometry gating strategy for determining the amount of antibody bound to the surface of an APC and the amount of total antibody associated with the APC.

FIG. 4 displays a graph showing the external staining of the APCs as compared to the total staining of the APCs for antibodies bococizumab and AVASTIN®.

FIG. 5 displays the internalization index values of different anti-PCSK9 antibodies that have different levels of immunogenicity in a clinical setting.

FIG. 6 displays the internalization index values of antibodies with high immunogenicity and antibodies with low immunogenicity in a clinical setting including ENBREL® (etanercept), HUMIRA® (adalimumab) and REMICADE® (infliximab) and the internalization index values of antibody aggregates.

FIG. 7A shows the internalization index values of antibodies bococizumab, adalimumab and bevacizumab over a time period of 72 hours.

FIG. 7B shows the internalization index values of antibodies bococizumab, adalimumab and bevacizumab using immature dendritic cells (iDCs) and dendritic cells that were stimulated with LPS to mature (mDCs).

FIG. 8 shows the internalization rates of bococizumab in the presence of cytochalasin, an Fc receptor block or a combination of both.

### DETAILED DESCRIPTION

For clarity, but not by way of limitation, the detailed description of the presently disclosed subject matter is divided into the following subsections:

I. Definitions;

II. Methods;

III. Polypeptides;

IV. Kits; and

V. Exemplary Embodiments.

### I. DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as ±10% of the value modified by the term “about.”

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab)₂; diabodies; linear antibodies; single-chain antibody molecules (e.g., scFv); and multispecific antibodies formed from antibody fragments.

An antibody “which binds” an antigen of interest is one that binds the antigen with sufficient affinity such that the antibody is useful as an assay reagent, e.g., as a detection antibody. Typically, such an antibody does not significantly cross-react with other polypeptides. With regard to the binding of a polypeptide to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a target molecule compared to binding of a control molecule, which generally is a molecule of similar structure that does not have binding activity.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). Affinity can be measured by common methods known in the art, including those described herein. Specific illustrative and exemplary embodiments for measuring binding affinity are described in the following.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “detection antibody,” as used herein, refers to an antibody that specifically binds a target molecule in a sample. Under certain conditions, the detection antibody forms a complex with the target molecule. A detection antibody is capable of being detected either directly through a label, which may be detected, or indirectly, e.g., through use of another antibody that is labeled and that binds the detection antibody. For direct labeling, the detection antibody is typically conjugated to a moiety that is detectable by some means, for example, including but not limited to, fluorophore.

The term “detecting,” is used herein, to include both qualitative and quantitative measurements of a target molecule or processed forms thereof. In certain embodiments, detecting includes identifying the mere presence of the target molecule as well as determining whether the target molecule is present at detectable levels.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain that contains at least a portion of the constant region. The term includes native sequence Fc regions and variant Fc regions. In certain embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (CDR) residues. The FR of a variable domain generally consists of four FR domains: FR1, FR2, FR3, and FR4. Accordingly, the CDR and FR sequences generally appear in the following sequence in VH (or VL): FR1-H1(L1)-FR2-H2(L2)-FR3-H3 (L3)-FR4.

The terms “full-length antibody,” “intact antibody” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In certain embodiments, for the VL, the subgroup is subgroup kappa I as in Kabat et al., supra. In certain embodiments, for the VH, the subgroup is subgroup III as in Kabat et al., supra.

A “humanized” antibody refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “CDR,” as used herein, refers to each of the regions of an antibody variable domain which are hypervariable in sequence (also referred to herein as “complementarity determining regions” or “CDRs”) and/or form structurally defined loops (“hypervariable loops”) and/or contain the antigen-contacting residues (“antigen contacts”). Unless otherwise indicated, CDR residues and other residues in the variable domain (e.g., FR residues) are numbered herein according to Kabat et al., supra. Generally, antibodies comprise six CDRs: three in the VH (H1, H2, H3), and three in the VL (L1, L2, L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including CDR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

An “immunoconjugate” refers to an antibody conjugated to one or more heterologous molecule(s), including but not limited to a cytotoxic agent.

An “individual,” “subject” or “donor” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. In certain embodiments, the individual, subject or donor is a human.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

An “isolated” antibody is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The terms “label” or “detectable label,” as used herein, refers to any chemical group or moiety that can be linked to a substance that is to be detected or quantitated, e.g., an antibody. A label is a detectable label that is suitable for the sensitive detection or quantification of a substance. Non-limiting examples of detectable labels include, but are not limited to, luminescent labels, e.g., fluorescent, phosphorescent, chemiluminescent, bioluminescent and electrochemiluminescent labels, radioactive labels, enzymes, particles, magnetic substances, electroactive species and the like. Alternatively, a detectable label can signal its presence by participating in specific binding reactions. Non-limiting examples of such labels include haptens, antibodies, biotin, streptavidin, his-tag, nitrilotriacetic acid, glutathione S-transferase, glutathione and the like.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the present disclosure in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler et al., Nature Medicine 2017, published online 12 Jun. 2017, doi:10.1038/nm.4356 or EP 2 101 823 B1).

“Purified” polypeptide (e.g., antibody), as used herein, refers to a polypeptide that has been increased in purity, such that it exists in a form that is more pure than it exists in its natural environment and/or when initially synthesized and/or amplified under laboratory conditions. Purity is a relative term and does not necessarily mean absolute purity.

The term “package insert,” as used herein, refers to instructions customarily included in commercial packages that contain information concerning the use of the components of the package.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “polypeptide” and “protein,” as used interchangeably herein, refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein,” as used herein, specifically encompass antibodies.

As used herein, the term “recombinant protein” refers generally to peptides and proteins that have been genetically manipulated. In certain embodiments, such recombinant proteins are “heterologous,” i.e., foreign to the cell being utilized.

A “sample,” as used herein, refers to a small portion of a larger quantity of material. In certain embodiments, a sample includes, but is not limited to, cells in culture, cell supernatants, cell lysates, serum, blood plasma, biological fluid (e.g., blood, plasma, serum, stool, urine, lymphatic fluid, ascites, ductal lavage, saliva and cerebrospinal fluid) and tissue samples. The source of the sample may be solid tissue (e.g., from a fresh, frozen, and/or preserved organ, tissue sample, biopsy or aspirate), blood or any blood constituents, bodily fluids (such as, e.g., urine, lymph, cerebral spinal fluid, amniotic fluid, peritoneal fluid or interstitial fluid), or cells from the individual, including circulating cells.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### II. METHODS

The presently disclosed subject matter provides methods for determining the propensity of therapeutic, e.g., a polypeptide or a fragment thereof, to elicit production of anti-drug antibodies (ADAs). In certain embodiments, the presently disclosed methods can be used to determine the propensity of an antibody or fragment thereof or an antibody-drug conjugate (ADC) to elicit production of ADAs. The present disclosure is based, in part, on the discovery that the internalization of an antibody by an antigen presenting cell (APC) correlates with previously reported clinical ADA rates of the antibody. The present disclosure also provides kits for performing the methods disclosed herein.

In certain embodiments, the methods of the present disclosure can be used to identify polypeptide variants, e.g., antibody variants, that have a reduced propensity to elicit production of ADAs compared to the parent polypeptide, e.g., a parent antibody. In certain embodiments, the methods disclosed herein can be used to analyze newly-developed polypeptides, e.g., antibodies. For example, but not by way of limitation, the methods disclosed herein can be used to identify a polypeptide, e.g., an antibody, that has a lower propensity to elicit ADAs from a larger repertoire of polypeptides that specifically bind to the same antigen. Alternatively and/or additionally, the methods disclosed herein can be used to identify a polypeptide, e.g., an antibody, that has a lower propensity to elicit ADAs compared to a commercially available or clinically tested polypeptide, e.g., an antibody, that binds to the same antigen. In certain embodiments, methods of the present disclosure can be used to determine the immunogenic potential of a newly developed polypeptide, e.g., an antibody, prior to clinical studies. In certain embodiments, the presently disclosed methods can be used to determine the immunogenicity potential of aggregates of a polypeptide, e.g., antibody. In certain embodiments, the presently disclosed methods can be used to analyze the immunogenicity of sequence variants of an antibody, e.g., those that can arise during the manufacture and/or production of the antibody.

In certain embodiments, methods of the present disclosure can include contacting an APC with a polypeptide or a composition comprising the polypeptide. In certain embodiments, the APCs are cultured with the polypeptide for an amount of time sufficient for internalization of the polypeptide. For example, but not by way of limitation, the APCs can be cultured in the presence of the polypeptide, e.g., for about 1 to about 72 hours. In certain embodiments, the APCs can be cultured in the presence of the polypeptide from about 12 to about 72 hours, about 12 to about 60 hours, about 12 hours to about 48 hours, about 12 hours to about 24 hours, about 24 hours to about 72 hours, about 24 hours to about 60 hours, about 24 to about 48 hours, about 48 hours to about 72 hours or about 48 hours to about 60 hours. In certain embodiments, the APCs can be cultured in the presence of the polypeptide from about 24 to about 48 hours. In certain embodiments, the APCs can be cultured in the presence of the polypeptide for about 72 hours or less. In certain embodiments, the APCs can be cultured in the presence of the polypeptide for about 60 hours or less. In certain embodiments, the APCs can be cultured in the presence of the polypeptide for about 48 hours or less. In certain embodiments, the APCs can be cultured in the presence of the polypeptide for about 36 hours or less. In certain embodiments, the APCs can be cultured in the presence of the polypeptide for about 24 hours or less.

In certain embodiments, the number of APCs used in a method disclosed herein can be from about 1×10⁵ to about 1×10⁷ cells, e.g., about 1×10⁶ cells. For example, but not by way of limitation, the number of APCs used can be from about 2×10⁵ to about 9×10⁶ cells, from about 3×10⁵ to about 8×10⁶ cells, from about 3×10⁵ to about 7×10⁶ cells, from about 4×10⁵ to about 6×10⁶ cells, from about 5×10⁵ to about 5×10⁶ cells, from about 6×10⁵ to about 4×10⁶ cells, from about 7×10⁵ to about 3×10⁶ cells, from about 8×10⁵ to about 2×10⁶ cells, from about 9×10⁵ to about 2×10⁶ cells or from about 9×10⁵ to about 1×10⁶ cells. In certain embodiments, about 1×10⁶ cells APCs are used.

In certain embodiments, the polypeptide can be used at a concentration from about 25 μg/ml to about 200 μg/ml, e.g., from about 25 μg/ml to about 100 μg/ml. For example, but not by way of limitation, the polypeptide can be used at a concentration from about 30 μg/ml to about 100 μg/ml, from about 35 μg/ml to about 100 μg/ml, from about 40 μg/ml to about 100 μg/ml, from about 45 μg/ml to about 100 μg/ml, from about 50 μg/ml to about 100 μg/ml, from about 55 μg/ml to about 100 μg/ml, from about 60 μg/ml to about 100 μg/ml, from about 65 μg/ml to about 100 μg/ml, from about 70 μg/ml to about 100 μg/ml, from about 75 μg/ml to about 100 μg/ml, from about 80 μg/ml to about 100 μg/ml, from about 85 μg/ml to about 100 μg/ml, from about 90 μg/ml to about 100 μg/ml, from about 95 μg/ml to about 100 μg/ml, from about 25 μg/ml to about 95 μg/ml, from about 25 μg/ml to about 90 μg/ml, from about 25 μg/ml to about 85 μg/ml, from about 25 μg/ml to about 80 μg/ml, from about 25 μg/ml to about 75 μg/ml, from about 25 μg/ml to about 70 μg/ml, from about 25 μg/ml to about 65 μg/ml, from about 25 μg/ml to about 60 μg/ml, from about 25 μg/ml to about 55 μg/ml, from about 25 μg/ml to about 50 μg/ml, from about 25 μg/ml to about 45 μg/ml, from about 25 μg/ml to about 40 μg/ml, from about 25 μg/ml to about 35 μg/ml, from about 25 μg/ml to about 30 μg/ml, from about 30 μg/ml to about 90 μg/ml, from about 40 μg/ml to about 80 μg/ml or from about 50 μg/ml to about 70 μg/ml.

APCs for use in the presently disclosed methods include any cell that can display an antigen complexed with a major histocompatibility complex (MHC) on its surface. For example, but not by way of limitation, the APCs can be dendritic cells, macrophages, monocytes, neutrophils and B cells. In certain embodiments, the APCs are dendritic cells. In certain embodiments, the dendritic cells can be immature dendritic cells. In certain embodiments, immature dendritic cells can be differentiated from monocytes. Non-limiting sources of monocytes include monocytic cell lines and primary monocytes isolated from donors. In certain embodiments, the monocytes are isolated from a sample of a donor, e.g., from a blood sample of the donor. Monocytes can be isolated from a sample of a donor by any method known in the art, e.g., by a density gradient such as a Ficoll density gradient. In certain embodiments, peripheral blood mononuclear cells (PBMCs) are initially isolated from a sample of a donor and then subjected to a CD14+ selection to isolate monocytes. In certain embodiments, the isolated monocytes are differentiated in the presence of differentiation factors, e.g., interleukin-4 (IL-4) and/or granulocyte-macrophage colony-stimulating factor (GM-CSF), to generate immature dendritic cells for use in the presently disclosed methods. For example, but not by way of limitation, isolated monocytes can be differentiated in the presence of about 0.1 ng/ml to about 10 ng/ml of IL-4, e.g., about 3 ng/ml of IL-4, and/or in the presence of about 1 ng/ml to about 100 ng/ml of GM-CSF, e.g., about 50 ng/ml of GM-CSF. Alternatively, dendritic cells, e.g., immature dendritic cells, for use in the present disclosed methods can be differentiated from stem cells or iPSC cells. See, e.g., Li et al., World J. Stem Cells 6(1):1-10 (2014), the contents of which are incorporated by reference herein in their entirety.

In certain embodiments, the APC is contacted with the polypeptide in the presence of an agent. In certain embodiments, the APC can be contacted with the polypeptide in the presence of an agent that induces the maturation of immature APCs into mature APCs. For example, but not by way of limitation, immature dendritic cells can be contacted with the polypeptide in the presence of an agent that induces the maturation of the immature dendritic cells into mature dendritic cells. In certain embodiments, the agent is a cytokine, e.g., an inflammatory cytokine. Non-limiting examples of inflammatory cytokines include TNFα, IL-6 and IL-1β. Additional non-limiting examples of inflammatory cytokines are provided in Turner et al., Biochimica et Biophysica Acta 1843(11):2563-2582 (2014), the contents of which are incorporated by reference herein in their entirety. Alternatively or additionally, the agent is a protein and/or compound that is induced by inflammatory cytokines, e.g., prostaglandin E2 (PGE2), or induces expression of inflammatory cytokines, e.g., lipopolysaccharide (LPS). In certain embodiments, the agent is lipopolysaccharide. In certain embodiments, the agent can be used at a concentration from about 0.001 ng/ml to about 1,000 ng/ml, e.g., from about 0.01 ng/ml to about 1,000 ng/ml, from about 0.1 ng/ml to about 1,000 ng/ml, from about 1 ng/ml to about 1,000 ng/ml, from about 10 ng/ml to about 1,000 ng/ml, from about 100 ng/ml to about 1,000 ng/ml, from about 0.001 ng/ml to about 100 ng/ml, from about 0.001 ng/ml to about 10 ng/ml, from about 0.001 ng/ml to about 1 ng/ml, from about 0.001 ng/ml to about 0.1 ng/ml or from about 0.001 ng/ml to about 0.01 ng/ml. In certain embodiments, the agent, e.g., LPS, can be used at a concentration of about 1 μg/ml. In certain embodiments, the APC is not contacted with an agent, e.g., LPS, in the presence of the polypeptide. In certain embodiments, the APC is an immature dendritic cell that has not been cultured in the presence of an agent, e.g., LPS.

In certain embodiments, the method can further include measuring the amount of polypeptide present on an outer surface of the APC. For example, but not by way of limitation, the method can include measuring the amount of polypeptide that is bound to the surface, e.g., plasma membrane, of the APC. In certain embodiments, the polypeptide can be associated with, e.g., bound to, a major histocompatibility complex (MHC) molecule on the surface of the APC. In certain embodiments, the polypeptide can be associated with, e.g., bound to, an MHC class II (MHCII) molecule on the surface of the APC. In certain embodiments, measuring the amount of polypeptide present on the outer surface of the APC can include (i) contacting the APC with a detection agent that binds to the polypeptide and (ii) determining the amount of detection agent that is bound to the polypeptide present on the outer surface of the APC. In certain embodiments, the APC is not permeabilized prior to contacting the APC with the detection agent to prevent the detection of the polypeptide that is within the APC, e.g., present within intracellular structures within the APC.

In certain embodiments, the method can further include measuring the total amount of polypeptide associated with the APC. In certain embodiments, the total amount of polypeptide associated with the APC includes the amount of polypeptide present on the outer surface of the APC and the amount of the polypeptide present within the APC. In certain embodiments, measuring the total amount of polypeptide associated with the APC includes (i) permeabilizing the APC, (ii) contacting the APC with a detection agent that binds to the polypeptide and (iii) determining the amount of detection agent that is bound to the polypeptide present on the outer surface of the APC and within the APC. Permeabilizing the APC allows the detection of the polypeptide that is present within the APC, e.g., present within intracellular structures within the APC. In certain embodiments, permeabilization allows the detection of the polypeptide that is present within endosomal and lysosomal structures within the APC. In certain embodiments, the APCs can be permeabilized using any technique known in the art, including but not limited to, using detergents or organic solvents. Non-limiting examples of permeabilization techniques are disclosed in Jamur and Oliver, Methods Mol. Biol. 588:63-66 (2010), the contents of which are hereby incorporated by reference herein in their entirety. In certain embodiments, the APCs are permeabilizing by contacting the APCs with a permeabilization agent. For example, but not by way of limitation, the permeabilization agent can be a detergent, e.g., saponin.

In certain embodiments, the amount of polypeptide present on an outer surface of the APC and/or the total amount of polypeptide associated with the APC can be measured between about 4 hours to about 72 hours after initial contact of the APCs with the polypeptide. For example, but not by way of limitation, the amount of polypeptide present on an outer surface of the APC and/or the total amount of polypeptide associated with the APC can be measured between about 4 hours to about 48 hours, between about 4 hours to about 24 hours, between about 4 hours to about 12 hours or between about 4 hours to about 8 hours after initial contact of the APCs with the polypeptide. In certain embodiments, the amount of polypeptide present on an outer surface of the APC and/or the total amount of polypeptide associated with the APC can be measured at about 4 hours after initial contact of the APCs with the polypeptide.

In certain embodiments, the detection agent for use in the methods disclosed herein is an antibody (also referred to herein as “a detection antibody”). In certain embodiments, the detection agent specifically binds to the polypeptide analyzed by the disclosed methods, e.g., the detection agent specifically binds to an epitope present on the polypeptide or fragment thereof. In certain embodiments, the detection agent is an antibody that binds to a specific class or subclass of antibody. For example, but not by way of limitation, the detection antibody can be an anti-IgG antibody if an IgG antibody is being analyzed by the methods disclosed herein. In certain embodiments, the detection agent used in the assay methods disclosed herein can be used at a concentration from about 1 μg/ml to about 50 μg/ml, from about 1 μg/ml to about 40 μg/ml, from about 1 μg/ml to about 30 μg/ml, from about 1 μg/ml to about 20 μg/ml or about 1 μg/ml to about 10 μg/ml. In certain embodiments, the detection agent is used at a concentration from about 1 μg/ml to about 10 μg/ml, e.g., at about 6 μg/ml.

In certain embodiments, the detection agents, e.g., detection antibodies, for use in the disclosed methods can be labeled. Labels include, but are not limited to, labels or moieties that are detected directly, such as fluorescent, chromophoric, electron-dense, chemiluminescent, and radioactive labels, as well as moieties, such as enzymes or ligands, that are detected indirectly, e.g., through an enzymatic reaction or molecular interaction. Non-limiting examples of labels include the radioisotopes ³²P, ¹⁴C, ¹²⁵I, ³H and ¹³¹I, fluorophores such as rare earth chelates or fluorescein and its derivatives, rhodamine and its derivatives, dansyl, umbelliferone, luciferases, e.g., firefly luciferase and bacterial luciferase (see U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase or microperoxidase, biotin/avidin, spin labels, bacteriophage labels, stable free radicals and the like. In certain embodiments, the detection agent, e.g., antibody, is labeled with a fluorophore.

In certain embodiments, determining the amount of detection agent that is bound to the polypeptide is performed by any method that is able to detect the detection agent. In certain embodiments, the detection agent can be detected by monitoring the label of the detection agent, e.g., the fluorescent label. For example, but not by way of limitation, the amount of detection agent that is bound to the polypeptide or fragment thereof is determined by detecting the fluorescence of the detection agent. In certain embodiments, determining the amount of detection agent that is bound to the polypeptide or fragment thereof is performed by flow cytometry.

In certain embodiments, the method further includes determining the amount of polypeptide that is internalized by the APC. For example, but not by way of limitation, the method can include calculating an internalization index value (also referred to herein as “the normalized MFI”), which correlates with the amount of polypeptide or fragment thereof that is internalized by the APC. In certain embodiments, the internalization index value is determined by subtracting the amount of polypeptide bound to the outer surface of the APC from the total amount of polypeptide associated with the APC.

In certain embodiments, the method can further include comparing the internalization index of a polypeptide to a reference internalization index indicative of a known propensity to elicit the production of ADAs, e.g., in the clinical setting. In certain embodiments, when the internalization index value of a polypeptide is greater than the reference internalization index, the polypeptide has a greater propensity to elicit production of ADAs than the reference, e.g., in the clinical setting. Alternatively, when the internalization index value of a polypeptide is less than the reference internalization index, the polypeptide has a lesser propensity to elicit production of ADAs than the reference, e.g., in the clinical setting.

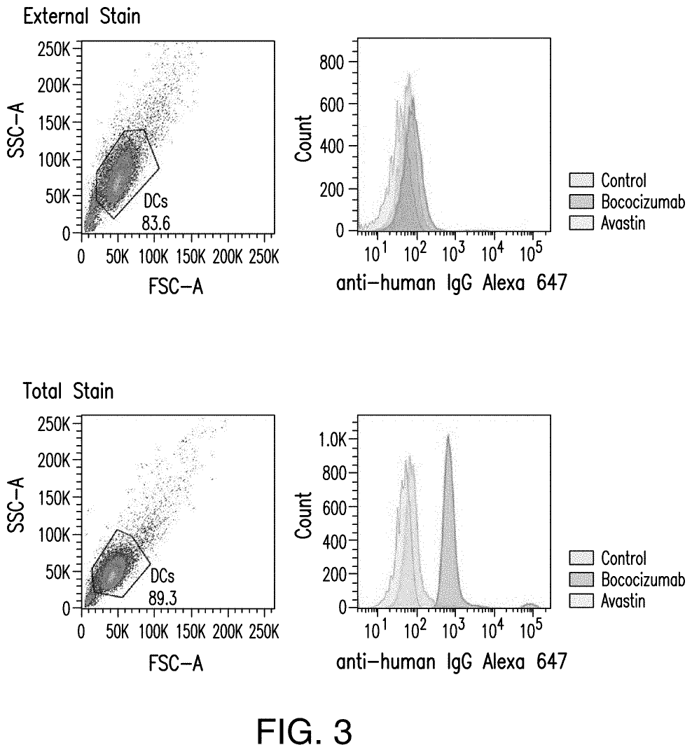

In certain embodiments, the reference internalization index can be the internalization index of a polypeptide (e.g., reference polypeptide) that has been shown to elicit production of ADAs. For example, but not by way of limitation, the reference polypeptide can be an antibody that has been shown to elicit production of ADAs in a clinical setting. In certain embodiments, reference polypeptides which have higher internalization index values have higher level of ADA/immunogenicity in a clinic setting. In certain embodiments, reference polypeptides with lower internalization index values have decreased ADA/immunogenicity in the clinic setting. In certain embodiments, the reference polypeptide can be an antibody disclosed in any one of FIGS. 3-8. In certain embodiments, the reference polypeptide can be an antibody that has been shown to elicit production of ADAs at a low level in a clinical setting, e.g., evolocumab, RG7652 and AVASTIN®. In certain embodiments, the reference polypeptide can be an antibody that has been shown to elicit the production of ADAs at a high level in a clinical setting, e.g., bococizumab. Alternatively or additionally, in the context of antibody variants, the reference internalization index can be the internalization index of the parent antibody. In certain embodiments, in the context of aggregates, the reference internalization index can be the internalization index of the same antibody that is not aggregated. In certain embodiments, the reference internalization index can be the internalization index of an antibody that binds the same antigen as the antibody being analyzed using the methods disclosed herein.

In certain embodiments, the method can include analyzing the polypeptide with dendritic cells obtained from more than one donor. In certain embodiments, a method of the present disclosure can include analyzing the propensity of a polypeptide to elicit production of ADAs by separately culturing dendritic cells, e.g., immature dendritic cells, derived from individual donors with the polypeptide of interest and analyzing the internalization index of dendritic cells of each individual donor. In certain embodiments, internalization index values for each of the individual donors can be determined and analyzed to determine the propensity of the polypeptide to elicit ADA production. In certain embodiments, the internalization index value can be an average of the internalization index values of every donor. In certain embodiments, dendritic cells derived from at least 2 or more, at least 3 or more, at least 4 or more, at least 5 or more, at least 6 or more, at least 7 or more, at least 8 or more, at least 9 or more, at least 10 or more, at least 15 or more, at least 20 or more, at least 25 or more, at least 30 or more, at least 35 or more or at least 40 or more individual donors can be separately cultured with the polypeptide of interest. In certain embodiments, dendritic cells from about 10 to about 50 individual donors can be separately cultured with the polypeptide of interest. For example, but not by way of limitation, dendritic cells from about 15 to about 45 individual donors, from about 20 to about 40 individual donors, from about 25 to about 35 individual donors or from about 30 individual donors can be separately cultured with the polypeptide of interest.

### III. POLYPEPTIDES

The present disclosure provides methods for determining the propensity of a polypeptide to elicit production of ADAs. Non-limiting examples of such polypeptides that can be analyzed by the disclosed methods are provided below. For example, but not by way of limitation, the polypeptide that is assayed using any of the methods disclosed herein can be a fragment of the polypeptide, e.g., a peptide. In certain embodiments, the polypeptide can be a recombinant protein. In certain embodiments, the polypeptide is an antibody or a fragment thereof, e.g., a human, humanized or chimeric antibody. In certain embodiments, the antibody can be an antibody-drug conjugate (ADC). In certain embodiments, the antibody can be a single domain antibody.

1. Antibodies or Fragments Thereof

In certain embodiments, a polypeptide analyzed by the methods disclosed herein is an antibody or a fragment thereof, e.g., monoclonal antibodies and fragments thereof. For example, but not by way of limitation, the methods of the present disclosure can be used to determine the immunological potential of newly developed and/or identified antibodies or fragments thereof.

Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For a discussion of Fab and F(ab′)₂ fragments comprising salvage receptor binding epitope residues and having increased in vivo half-life, see U.S. Pat. No. 5,869,046. Antibody fragments can be made by various techniques including, but not limited to, proteolytic digestion of an intact antibody as well as production by recombinant host cells (e.g., E. coli or phage), as described herein.

In certain embodiments, a polypeptide analyzed by the methods disclosed herein can be a diabody. Diabodies are antibody fragments comprising two antigen-binding sites that may be bivalent or bispecific. See, for example, EP 404097; WO 1993/01161; Hudson et al., Nat. Med. 9:129-134 (2003); and Hollinger et al., Proc. Natl. Acad. Sci. USA 90: 6444-6448 (1993). Triabodies and tetrabodies, which are also described in Hudson et al., Nat. Med. 9:129-134 (2003) can be analyzed by the disclosed methods.

In certain embodiments, the antibody analyzed by the disclosed methods can be a single-domain antibody. Single-domain antibodies are antibody fragments that comprise all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). Additional non-limiting examples of single-domain antibodies are disclosed in Iezzi et al., Front Immunol. 9:273 (2018), the contents of which are incorporated by reference herein in their entirety. In certain embodiments, the antibody is a hybribody (Hybrigenics Services, Cambridge, Mass.).

2. Chimeric, Humanized and Human Antibodies

In certain embodiments, a polypeptide analyzed by the methods disclosed herein is a chimeric antibody, e.g., a humanized antibody. For example, but not by way of limitation, the presently disclosed methods can be used to identify chimeric versions of an antibody that have a low or lower propensity to elicit productions of ADAs, e.g., compared to the parent antibody or other chimeric versions of the antibody. Alternatively or additionally, methods of the present disclosure can be used to identify chimeric antibodies that have a low propensity to elicit production of ADAs.

Certain chimeric antibodies are described in the art, e.g., in U.S. Pat. No. 4,816,567; and Morrison et al., Proc. Natl. Acad. Sci. USA, 81:6851-6855 (1984)). In certain embodiments, a chimeric antibody includes a non-human variable region (e.g., a variable region derived from a mouse, rat, hamster, rabbit or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In certain embodiments, a chimeric antibody can be a humanized antibody. Typically, a non-human antibody is humanized to reduce immunogenicity to humans, while retaining the specificity and affinity of the parental non-human antibody. Generally, a humanized antibody comprises one or more variable domains in which CDRs, e.g., CDRs, (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the CDR residues are derived), e.g., to restore or improve antibody specificity or affinity.

In certain embodiments, a polypeptide analyzed by the methods disclosed herein can be a human antibody. For example, but not by way of limitation, the presently disclosed methods can be used to identify chimeric versions of an antibody that has a low or lower propensity to elicit productions of human antibodies that have a low propensity to elicit production of ADAs.

3. Library-Derived Antibodies

In certain embodiments, a polypeptide analyzed by the methods disclosed herein can be an antibody or fragment thereof isolated by screening combinatorial libraries for antibodies with the desired activity or activities. For example, but not by way of limitation, the presently disclosed methods can be used to identify a library-derived antibody that has a low or lower propensity to elicit productions of ADAs, e.g., as compared other library-derived antibodies that possess the desired binding characteristics and/or bind to the same antigen.

Antibodies or antibody fragments isolated from human antibody libraries are considered human antibodies or human antibody fragments herein.

4. Multispecific Antibodies

In certain embodiments, a polypeptide analyzed by the methods disclosed herein can be a multispecific antibody, e.g., a bispecific antibody. Multispecific antibodies are monoclonal antibodies that have binding specificities for at least two different epitopes. Bispecific antibodies can be prepared as full-length antibodies or antibody fragments. For example, but not by way of limitation, the presently disclosed methods can be used to identify multispecific antibodies that have a low or lower propensity to elicit productions of ADAs, e.g., compared to other multispecific antibodies that bind the same epitopes. Alternatively or additionally, methods of the present disclosure can be used to identify multispecific antibodies that have a low propensity to elicit production of ADAs.

Engineered antibodies with three or more functional antigen binding sites, including “Octopus antibodies,” can also be analyzed by the disclosed methods (see, e.g., US 2006/0025576A1).

5. Immunoconjugates

In certain embodiments, a polypeptide analyzed by the methods disclosed herein can be an immunoconjugate, e.g., an immunoconjugate comprising an antibody conjugated to one or more cytotoxic agents, such as chemotherapeutic agents or drugs, growth inhibitory agents, toxins (e.g., protein toxins, enzymatically active toxins of bacterial, fungal, plant or animal origin, or fragments thereof) or radioactive isotopes. For example, an antibody or antigen-binding portion can be functionally linked (e.g., by chemical coupling, genetic fusion, noncovalent association or otherwise) to one or more other binding molecules, such as another antibody, antibody fragment, peptide or binding mimetic.

In certain embodiments, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In certain embodiments, an immunoconjugate comprises an antibody conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In certain embodiments, an immunoconjugate comprises an antibody conjugated to a radioactive atom to form a radioconjugate. A variety of radioactive isotopes are available for the production of radioconjugates. Non-limiting examples include At²¹¹, I¹³¹, I¹²⁵, Y⁹⁰, Re¹⁸⁶, Re¹⁸⁸, Sm¹⁵³, Bi²¹², P³², P²¹² and radioactive isotopes of Lu. When the radioconjugate is used for detection, it can include a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent can be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker can be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) can be used.

In certain embodiments, immunoconjugates include, but are not limited to, such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

6. Antibody Variants

In certain embodiments, a polypeptide analyzed by the methods disclosed herein can be an antibody variant of a previously disclosed antibody. For example, methods of the present disclosure can be used to identify antibodies that are variants of previously disclosed antibodies that have a lower propensity to elicit productions of ADAs, e.g., than the parent antibody. In certain embodiments, amino acid substitutions can be introduced into an antibody of interest and the antibody variants can be screened for immunogenicity by using the disclosed methods.

In certain embodiments, the antibody variant can be amino acid sequence variants of an antibody, e.g., prepared by introducing appropriate modifications into the nucleotide sequence encoding the antibody or by peptide synthesis. Such modifications include, but are not limited to, deletions from, and/or insertions into and/or substitutions of residues within the amino acid sequences of the antibody. Sites of interest for such variation include, but are not limited to, the CDRs, and FRs. Any combination of deletion, insertion, and substitution can be made to arrive at the final construct, provided that the final antibody, i.e., modified, possesses the desired characteristics, e.g., antigen-binding.

In certain embodiments, an antibody variant can be an antibody that has been altered to increase or decrease the extent to which the antibody is glycosylated. For example, but not by way of limitation, the addition or deletion of glycosylation sites of an antibody can be accomplished by altering the amino acid sequence such that one or more glycosylation sites is created or removed.

In certain embodiments, an antibody analyzed by the methods disclosed herein is an Fc region variant. The Fc region variant can include a human Fc region sequence (e.g., a human IgG1, IgG2, IgG3 or IgG4 Fc region) comprising an amino acid modification (e.g., a substitution) at one or more amino acid positions.

In certain embodiments, an antibody variant can be a cysteine engineered antibody, e.g., “thioMAb,” in which one or more residues of an antibody are substituted with cysteine residues. In certain embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies can be generated as described, e.g., in U.S. Pat. No. 7,521,541.

### IV. KITS

The presently disclosed subject matter further provides kits containing materials useful for performing the methods disclosed herein. In certain embodiments, a kit of the present disclosure includes a container containing APCs or monocytes and/or a container containing one or more detection agents. Non-limiting examples of suitable containers include bottles, test tubes, vials and microtiter plates. The containers can be formed from a variety of materials such as glass or plastic.

In certain embodiments, the kit can include one or more containers containing one or more APCs or monocytes. Non-limiting examples of APCs include dendritic cells, macrophages, monocytes and B cells. In certain embodiments, the kit can include at least one container containing dendritic cells. For example, but not by way of limitation, the kit can include at least one container that includes immature dendritic cells. In certain embodiments, a kit of the present disclosure includes APCs derived from one or more donors in one or more containers. For example, but not by way of limitation, APCs from each individual donor are provided in separate containers. In certain embodiments, a kit of the present disclosure can include APCs from about 10 to about 40 individual donors. In certain embodiments, a kit of the present disclosure can further include one or more detection agents. For example, but not by way of limitation, the detection agent can be an antibody that specifically binds to the polypeptide being analyzed, e.g., an anti-IgG antibody if an IgG antibody is being analyzed.

In certain embodiments, a kit of the present disclosure can further include one or more agents for inducing maturation of immature dendritic cells in one or more containers. In certain embodiments, the one or more agents can include inflammatory cytokines, PGE2 and lipopolysaccharide.

In certain embodiments, the kit further includes a package insert that provides instructions for using the components provided in the kit. For example, a kit of the present disclosure can include a package insert that provides instructions for using the APCs, monocytes and/or detection agents in the disclosed methods.

Alternatively or additionally, the kit can include other materials desirable from a commercial and user standpoint, including other buffers, diluents and filters. In certain embodiments, the kit can include materials for collecting and/or processing blood samples, e.g., to isolate PBMCs and/or monocytes from a blood sample. In certain embodiments, the kit can include reagents for differentiating monocytes into immature dendritic cells. In certain embodiments, the one or more agents for differentiating monocytes into immature dendritic cells can be IL-4 and/or GM-CSF. In certain embodiments, the kit can further include agents capable of permeabilizing APCs, e.g., saponin.

### V. EXEMPLARY EMBODIMENTS

A. In certain non-limiting embodiments, the presently disclosed subject matter provides for a method for determining the propensity of a polypeptide, or a fragment thereof, to elicit the production of anti-drug antibodies (ADAs) relative to a known reference, comprising:
(a) contacting an antigen presenting cell (APC) with the polypeptide, or fragment thereof;
(b) measuring the amount of polypeptide, or fragment thereof, present on an outer surface of the APC;
(c) measuring the total amount of polypeptide, or fragment thereof, associated with the APC, wherein the total amount of polypeptide, or fragment thereof, associated with the APC comprises the amount of polypeptide, or fragment thereof, present on the outer surface of the APC and the amount of the polypeptide, or fragment thereof, present within the APC;
(d) calculating an internalization index value by subtracting the amount of polypeptide, or fragment thereof, bound to the outer surface of the APC measured in (b) from the total amount of polypeptide, or fragment thereof, associated with the APC measured in (c); and
(e) comparing the internalization index in (d) to a reference internalization index;
wherein when the internalization index value in (d) is greater than the reference internalization index, the polypeptide, or fragment thereof, has a greater propensity to elicit ADAs than the reference and when the internalization index value in (d) is less than the reference internalization index, the polypeptide, or fragment thereof, has a lesser propensity to elicit ADAs than the reference.
A1. The method of A, wherein the polypeptide, or fragment thereof, is a peptide.
A2. The method of A, wherein the polypeptide, or fragment thereof, is a recombinant protein.
A3. The method of A, wherein the polypeptide, or fragment thereof, is an antibody or fragment thereof.
A4. The method of A3, wherein the antibody or fragment thereof is a human, humanized or chimeric antibody.
A5. The method of A3 or A4, wherein the antibody or fragment thereof is a single domain antibody.
A6. The method of A, wherein the polypeptide is an antibody-drug conjugate (ADC).
A7. The method of any one of A-A6, wherein the APC is selected from the group consisting of a dendritic cell, a macrophage, a monocyte and a B cell.
A8. The method of A7, wherein the APC is a dendritic cell.
A9. The method of A8, wherein the dendritic cell is an immature dendritic cell.
A10. The method of A9, wherein the immature dendritic cell is generated by differentiating monocytes isolated from a donor.
A11. The method of A10, wherein the isolated monocytes are differentiated in the presence of one or more of interleukin-4 (IL-4) and granulocyte-macrophage colony-stimulating factor (GM-CSF) to generate the immature dendritic cells.
A12. The method of any one of A-A11, wherein the APC is contacted with the polypeptide, or fragment thereof, in the presence of an agent.
A13. The method of A12, wherein the agent is selected from the group consisting of an inflammatory cytokine, prostaglandin E2 (PGE2), lipopolysaccharide (LPS) and a combination thereof.
A14. The method of A13, wherein the inflammatory cytokine is selected from the group consisting of TNFα, IL-6, IL-1β and a combination thereof.
A15. The method of A13, wherein the agent is LPS.
A16. The method of any one of A-A15, wherein (c) measuring the total amount of polypeptide, or fragment thereof, associated with the APC comprises:
(i) permeabilizing the APC;
(ii) contacting the APC with a detection agent that binds to the polypeptide or fragment thereof; and
(iii) determining the amount of detection agent that is bound to the polypeptide, or fragment thereof, present on the outer surface of the APC and within the APC to measure the total amount of polypeptide or fragment thereof, associated with the APC.
A17. The method of any one of A-A16, wherein (b) measuring the amount of polypeptide, or fragment thereof, present on the outer surface of the APC comprises:
(i) contacting the APC with a detection agent that binds to the polypeptide or fragment thereof; and
(ii) determining the amount of detection agent that is bound to the polypeptide or fragment thereof, present on the outer surface of the APC to measure the amount of polypeptide or fragment thereof, present on the outer surface of the APC, wherein the APC is not permeabilized prior to contacting the APC with the detection agent.
A18. The method of A16 or A17, wherein the detection agent is an antibody.
A19. The method of any one of A16-A18, wherein the detection agent is an antibody conjugated to a fluorophore.
A20. The method of A18 or A19, wherein the antibody is an anti-IgG antibody.
A21. The method of any one of A16-A20, wherein determining the amount of detection agent that is bound to the polypeptide, or fragment thereof, is performed by flow cytometry.
B. In certain non-limiting embodiments, the presently disclosed subject matter provides for a kit for performing any one of the methods of A-A21.
B1. The kit of B, wherein the kit comprising one or more of the following:
(a) an APC;
(b) an agent;
(c) a detection agent; and
(d) a permeabilization agent.
B2. The kit of B1, wherein the agent is selected from the group consisting of an inflammatory cytokine, prostaglandin E2 (PGE2), lipopolysaccharide (LPS) and a combination thereof.
B3. The kit of B1, wherein the inflammatory cytokine is selected from the group consisting of TNFα, IL-6, IL-1β and a combination thereof.
B4. The kit of B1 or B2, wherein the agent is LPS.
B5. The kit of any one of B1-B4, wherein the detection agent is an antibody.
B6. The kit of any one of B1-B5, wherein the detection agent is an antibody conjugated to a fluorophore.
B7. The kit of B5 or B6, wherein the antibody is an anti-IgG antibody.
B8. The kit of any one of B1-B7, wherein the permeabilization agent is saponin.

The following example is merely illustrative of the presently disclosed subject matter and should not be considered as limiting in any way.

### Example 1: Antigen Presenting Cell-Based Assay

Polypeptide-based therapeutics have the immunogenic potential to elicit the production of ADAs. In particular, such polypeptide-based therapeutics can be taken up and processed by antigen presenting cells such as dendritic cells to present fragments of the polypeptide-based therapeutic in complex with a class II MHC molecule on its surface. T cells subsequently interact with the fragments presented on the surface of the antigen presenting cells to elicit an immune reaction that results in the production of ADAs by B cells.

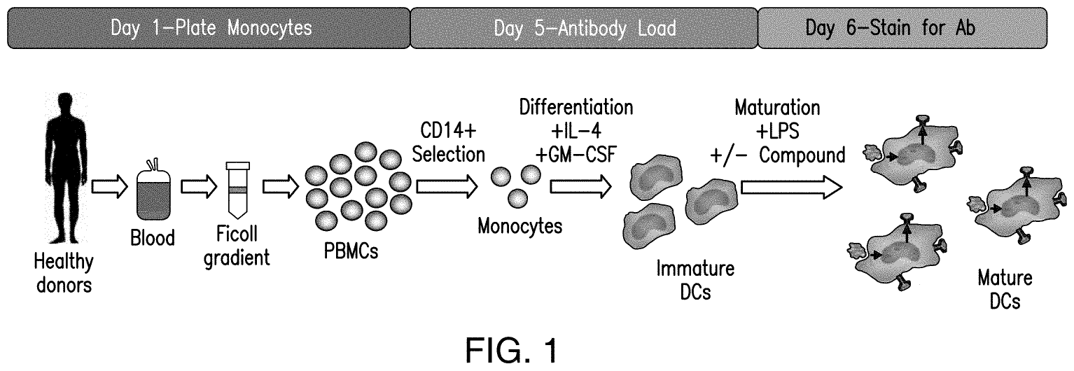

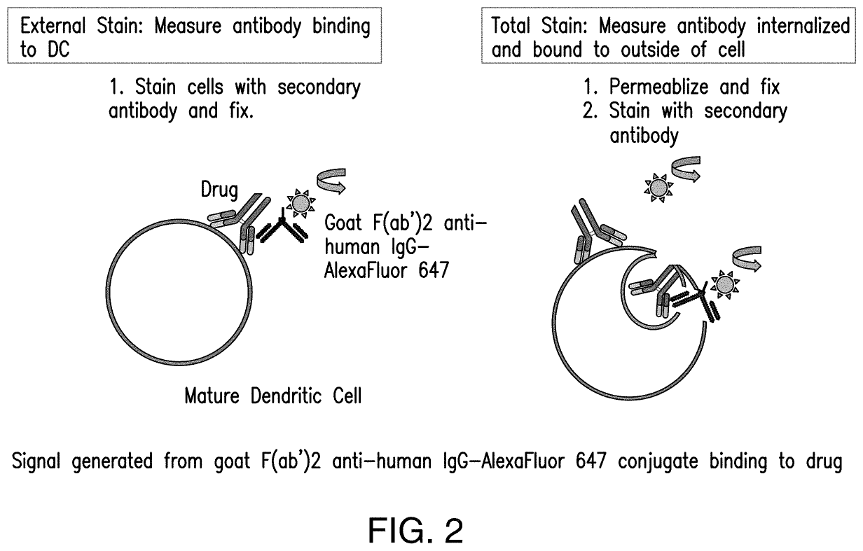

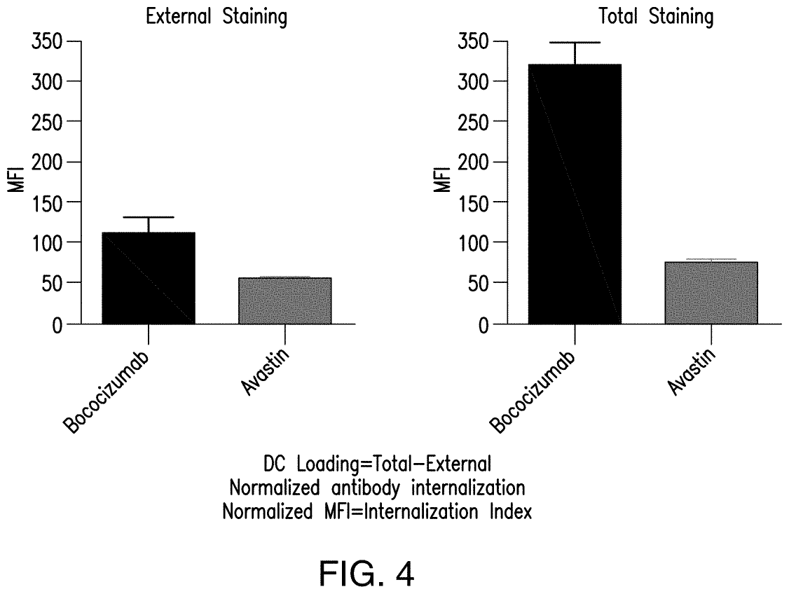

A method for determining the propensity of an antibody to elicit production of ADAs has been developed herein. Such a method can be an extremely valuable tool during drug development as it can be used to predict the immunogenic potential of a newly developed drug in the preclinical stage of development. FIG. 1 provides a schematic of the experimental details of the method. As shown in FIG. 1, PBMCs were isolated from the blood of a donor using a Ficoll gradient. Monocytes were isolated from the PBMCs via CD14+ beads (Miltenyi Biotec, following manufacturer's directions) and subsequently differentiated into immature dendritic cells by incubating the monocytes with 3 ng/mL IL-4 and 50 ng/mL GM-CSF (R&D Systems). At day 5 following the initial plating of the isolated monocytes and subsequent differentiation of the monocytes, the immature dendritic cells were contacted with 100 μg/mL of the antibody of interest in the presence of 1 μg/mL lipopolysaccharide (LPS) to promote maturation of the immature dendritic cells. On day 6, the mature dendritic cells with stained and analyzed according to the details provided in FIGS. 2-4. FIG. 2 displays the technique for determining the amount of antibody bound to the surface of an APC and the amount of total antibody associated with the APC. To determine the amount of antibody bound to the surface of the APC, the APCs were stained with 6 μg/mL of anti-human IgG antibody that is conjugated to AlexaFluor 647 (FIG. 2). To determine the amount of total antibody associated with the APCs, the APCs were fixed and permeabilized (Becton Dickinson Fixation and Permeabilization buffer, following manufacturer's directions) prior to staining with the anti-human IgG antibody conjugated to AlexaFluor 647 (FIG. 2). Permeabilization of the APCs allows the fluorophore-conjugated antibody to enter the cells and bind to antibody that is present within intracellular structures such as endosomes. The amount of antibody bound to the surface of the APC as compared to the amount of total antibody associated with the APC was determined by flow cytometry. FIG. 3 discloses the gating strategy used for the flow cytometry. To determine the internalization index (also referred to herein as “the normalized MFI”), the amount of antibody of interest that is bound to the APCs were subtracted from the total amount of the antibody of interest that is associated with the APCs (FIG. 4). As shown in FIG. 4, the antibody bococizumab, which has been shown to have a high clinical ADA rate, resulted in a greater amount of total antibody associated with APCs compared to AVASTIN®, which has been shown to have a lower clinical ADA rate. These results indicate that bococizumab would have a higher internalization index than AVASTIN®.

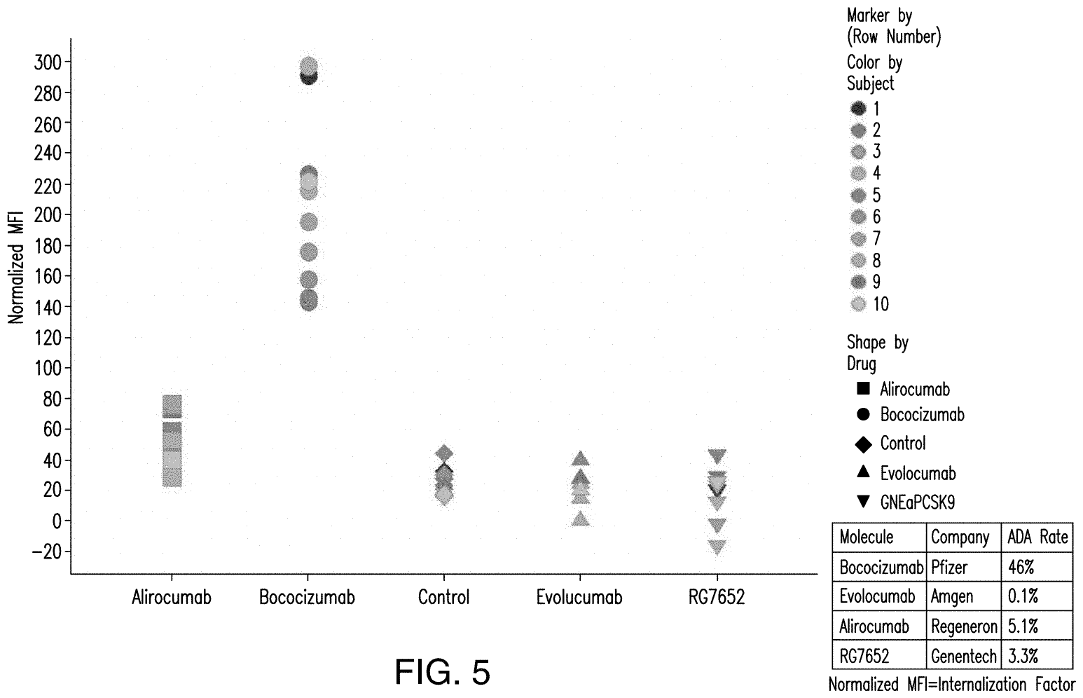

Analysis of a number of anti-PCSK9 antibodies confirmed that the internalization index correlates with the clinical ADA rate (FIG. 5). Anti-PCSK9 antibodies, alirocumab, bococizumab, evolocumab and RG7652, were analyzed by the method described above. These 4 different anti-PCSK9 antibodies show different levels of immunogenicity in the clinic. In particular, bococizumab has been shown to have a clinical ADA rate of 46%, evolocumab has been shown to have a clinical ADA rate of 0.1%, alirocumab has a clinical ADA rate of 5.1% and RG7652 has a clinical ADA rate of 3.3%. As shown in FIG. 5, the internalization index of bococizumab was significantly higher than any of the other anti-PCSK9 antibodies, which relates to the clinical ADA rates observed for the different antibodies.

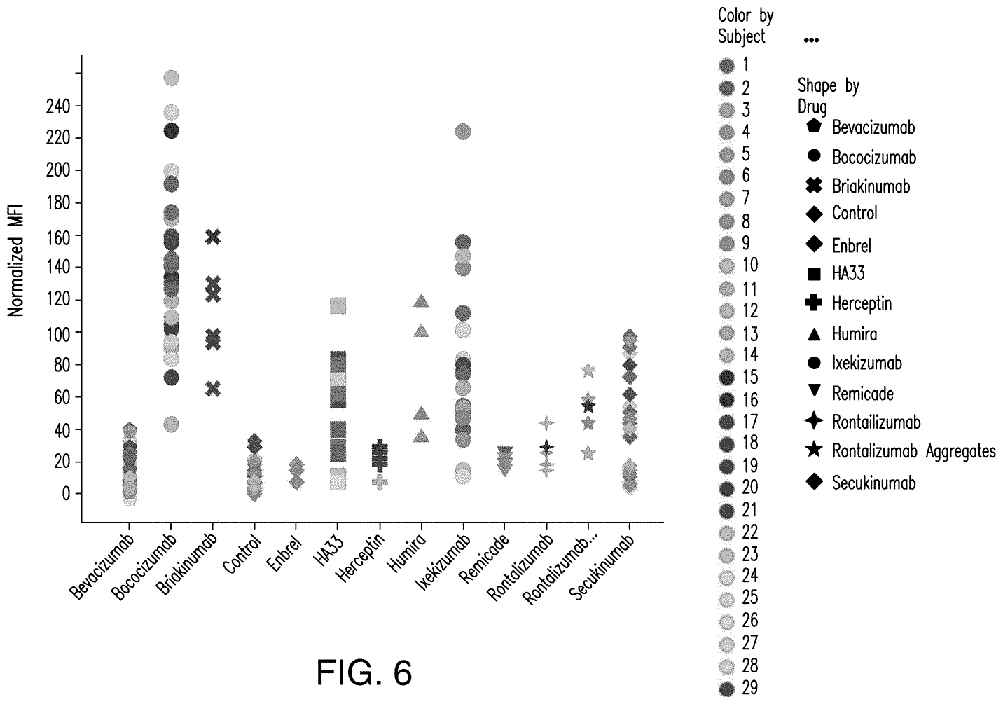

Analysis of a number of anti-TNF antibodies displayed similar results (FIG. 6). The anti-TNF antibodies ENBREL® (etanercept), HUMIRA® (adalimumab) and REMICADE® (infliximab) were analyzed. ENBREL®, HUMIRA® and REMICADE® have clinical ADA rates of 8.7%, 26% and 10%, respectively. As shown in FIG. 6, HUMIRA®, which has the highest clinical ADA, also had the highest internalization index (i.e., normalized MFI) as compared to ENBREL® and REMICADE®.

Further analysis of a number of additional antibodies showed that this method can distinguish between antibodies that have high immunogenicity and antibodies that have lower immunogenicity. As shown in FIG. 6, bococizumab, HA33 and briakinumab, which have high ADA rates resulted in high internalization indexes compared to HERCEPTIN®, which has a low internalization index. The ADA rates for rontalizumab and HERCEPTIN® are 0.6% and 10-15%, respectively. Secukinumab and ixekizumab, which both target IL-17a, were also analyzed. In the clinic, secukinumab has a low immunogenicity rate (less than 1%), but ixekizumab has a higher rate, from 5-22%. As shown in FIG. 6, ixekizumab internalized to a higher degree than secukinumab. These data show that the internalization index is higher for molecules that show high immunogenicity in the clinic (i.e., higher clinical ADA rate) and lower for molecules that show low immunogenicity in the clinic (i.e., lower clinical ADA rate). In addition, antibody aggregation, which is known to affect the immunogenicity of an antibody can be measured by the disclosed method. As shown in FIG. 6, aggregates of the antibody rontalizumab (referred to as “Rontalizumab . . . ” and “Rontalizumab Aggregates”) had a higher internalization index than rontalizumab.

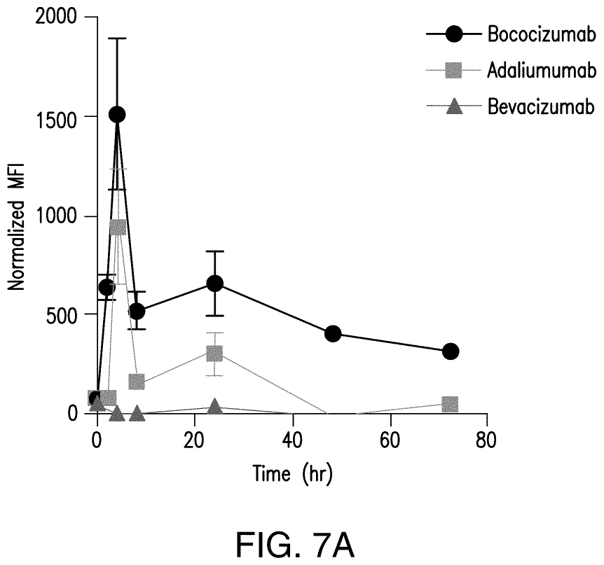

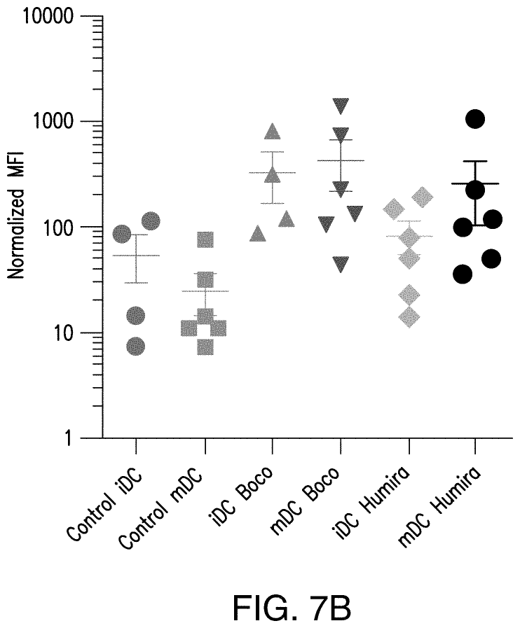

Additional experiments were performed to determine how the internalization indexes of bococizumab, adalimumab and bevacizumab change over time. The ADA rates for bococizumab and adalimumab are noted above and the ADA rate for bevacizumab is 0.6%. As shown in FIG. 7A, the greatest amount of antibody was internalized 4 hours after contacting the immature dendritic cells with the antibodies. After 48 or 72 hours, no further increase in antibody internalization was observed (FIG. 7A). Further analysis was performed to determine if the amount of antibody internalized depends on whether the dendritic cells are immature or mature. As shown in FIG. 7B, minimal differences in antibody internalization were observed in immature dendritic cells (iDCs) compared to dendritic cells that were stimulated with LPS to mature (mDCs).

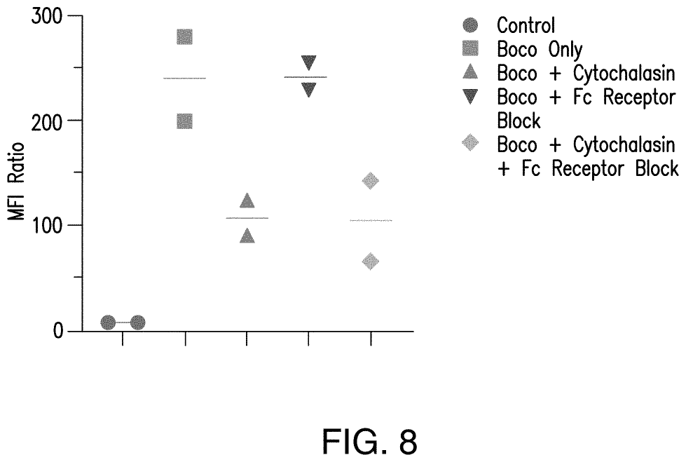

The mechanism by which dendritic cells internalize antibodies was determined by analyzing the internalization index of bococizumab in the presence of cytochalasin, an antibody cocktail composed of anti-FcγRIA, anti-FcγRIIa and anti-FcγRIIIA that blocks all of the FcγRs (also referred to as an Fc receptor block) or both. As shown in FIG. 8, the use of cytochalasin alone or in combination with the Fc receptor block inhibited the internalization of bococizumab compared to the control or the use of the Fc receptor block alone. These data indicate that the internalization of antibodies by dendritic cells is driven by macropinocytosis rather than through Fc receptor-mediated internalization.

The data shows that the disclosed method can be used for determining the immunogenicity of an antibody as it reproducibly distinguishes between antibodies with high clinical immunogenicity and antibodies with low clinical immunogenicity. The disclosed method is also cost-effective and does not require much equipment to perform.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various publications, patents and patent applications are cited herein, the contents of which are hereby incorporated by reference in their entireties.

What is claimed is:

1. A method for determining the propensity of a polypeptide, or a fragment thereof, to elicit the production of anti-drug antibodies (ADAs) relative to a known reference, comprising:
(a) contacting an antigen presenting cell (APC) with the polypeptide, or fragment thereof;
(b) measuring the amount of polypeptide, or fragment thereof, present on an outer surface of the APC;
(c) measuring the total amount of polypeptide, or fragment thereof, associated with the APC, wherein the total amount of polypeptide, or fragment thereof, associated with the APC comprises the amount of polypeptide, or fragment thereof, present on the outer surface of the APC and the amount of the polypeptide, or fragment thereof, present within the APC;
(d) calculating an internalization index value by subtracting the amount of polypeptide, or fragment thereof, bound to the outer surface of the APC measured in (b) from the total amount of polypeptide, or fragment thereof, associated with the APC measured in (c); and
(e) comparing the internalization index in (d) to a reference internalization index;
wherein when the internalization index value in (d) is greater than the reference internalization index, the polypeptide, or fragment thereof, has a greater propensity to elicit ADAs than the reference and when the internalization index value in (d) is less than the reference internalization index, the polypeptide, or fragment thereof, has a lesser propensity to elicit ADAs than the reference.

2. The method of claim 1, wherein the polypeptide, or fragment thereof, is a peptide or a recombinant protein.

3. The method of claim 1, wherein the polypeptide, or fragment thereof, is an antibody or fragment thereof.

4. The method of claim 1, wherein the polypeptide is an antibody-drug conjugate (ADC).

5. The method of claim 1, wherein the APC is selected from the group consisting of a dendritic cell, a macrophage, a monocyte and a B cell.

6. The method of claim 5, wherein the APC is a dendritic cell.

7. The method of claim 6, wherein the dendritic cell is an immature dendritic cell.

8. The method of claim 7, wherein the immature dendritic cell is generated by differentiating monocytes isolated from a donor.

9. The method of claim 1, wherein the APC is contacted with the polypeptide, or fragment thereof, in the presence of an agent.

10. The method of claim 9, wherein the agent is selected from the group consisting of an inflammatory cytokine, prostaglandin E2 (PGE2), lipopolysaccharide (LPS) and a combination thereof.

11. The method of claim 10, wherein the inflammatory cytokine is selected from the group consisting of TNFα, IL-6, IL-1β and a combination thereof.

12. The method of claim 10, wherein the agent is LPS.

13. The method of claim 1, wherein (c) measuring the total amount of polypeptide, or fragment thereof, associated with the APC comprises:
(i) permeabilizing the APC;
(ii) contacting the APC with a detection agent that binds to the polypeptide or fragment thereof; and
(iii) determining the amount of detection agent that is bound to the polypeptide, or fragment thereof, present on the outer surface of the APC and within the APC to measure the total amount of polypeptide or fragment thereof, associated with the APC.

14. The method of claim 1, wherein (b) measuring the amount of polypeptide, or fragment thereof, present on the outer surface of the APC comprises:
(i) contacting the APC with a detection agent that binds to the polypeptide or fragment thereof; and
(ii) determining the amount of detection agent that is bound to the polypeptide or fragment thereof, present on the outer surface of the APC to measure the amount of polypeptide or fragment thereof, present on the outer surface of the APC, wherein the APC is not permeabilized prior to contacting the APC with the detection agent.

15. The method of claim 13, wherein the detection agent is an antibody.

16. The method of claim 13, wherein determining the amount of detection agent that is bound to the polypeptide, or fragment thereof, is performed by flow cytometry.

17. A kit for performing the method of claim 1.

18. The kit of claim 17, wherein the kit comprises one or more of the following:
(a) an APC;
(b) an agent;
(c) a detection agent; and
(d) a permeabilization agent.

19. The kit of claim 18, wherein (i) the agent is selected from the group consisting of an inflammatory cytokine, prostaglandin E2 (PGE2), lipopolysaccharide (LPS) and a combination thereof; (ii) the detection agent is an antibody; and/or (iii) the permeabilization agent is saponin.

20. The kit of claim 19, wherein the agent is LPS.
